(19) United States
(12) Patent Application Publication
(10) Pub. No.: US 2026/0294489 A1
(43) Pub. Date: Oct. 1, 2026

(54) PELVIC FIXATION DEVICE

(71) Applicant: William Oxner, Halifax (CA)

(72) Inventor: William Oxner, Halifax (CA)

(21) Appl. No.: 19/489,073

(22) PCT Filed: May 29, 2024
(86) PCT No.: PCT/CA2024/050714

§ 371 Date: Dec. 1, 2025

Related U.S. Application Data
(60) Provisional application No. 63/504,775, filed on May 29, 2023.

(51) Int. Cl. A61B 17/70; A61B 17/56

(52) U.S. Cl. CPC A61B 17/7055; A61B 17/7032; A61B 2017/564

(57) ABSTRACT

A pelvic fixation device is provided herein. The pelvic fixation devices include a first fixation member pair, a second fixation member pair, and a connecting rod. The first fixation member pair includes a first main member and a first interlocking member. The first interlocking member is connected obliquely to the first main member to prevent rotation. The second fixation member pair includes a second main member and a second interlocking member. The second interlocking member is connected obliquely to the second main member to prevent rotation. The connecting rod is defined with two ends to securely connect with the proximal ends of the first fixation member pair and the second fixation member pair, thereby laterally stabilizing each half of the pelvis. Angular stable locking is achieved through the interlocked screw pairs. The connecting rod stabilized the pelvis while avoiding any trauma from violation of the sacroiliac joint.

### TECHNICAL FIELD

The following relates generally to medical devices, and more particularly to a device for fixing and stabilizing the pelvis as part of medical treatment.

### INTRODUCTION

Pelvic injuries commonly result from high-energy trauma and external force impacts such as automobile accidents or falls from an elevation. Based on the direction and force of impact, pelvic injuries such as ligamentous or osseous lesions, hemodynamic shock, pelvic ring fractures, and arterial haemorrhaging may be caused. Pelvic fractures may cause serious consequences such as high mortality, mobility restrictions, and morbidity. Further, pelvic fractures may lead to loss of stability and increased volume of intra-pelvic compartments.

Besides high-energy trauma, stable pelvic fractures or lateral compression injuries may occur in the elderly population due to low-impact events such as a minor fall or running.

Restoration of pelvic anatomy and stabilization of the restored pelvis using biomechanically strong and non-expandable fixators is essential in the treatment of pelvic injuries. Re-establishing the integrity of pelvic rings is necessary for the restoration of pelvic anatomy, in addition to measures to restrain blood loss and provide circulatory control.

Pelvic fixation techniques are used for the stabilization of the pelvis in the treatment of pelvic fractures resulting from high-energy trauma. Pelvic fixation is aimed at achieving an anatomical reduction and stable fixation of the fracture.

Pelvic fixators provide a stable base to maintain the surgical correction until a fusion is obtained. A variety of pelvic stabilization fixators including iliac screws, plates, and rods are used to stabilize the pelvis as part of the treatment. The stabilization fixators are thereafter tightened and locked allowing the broken bone fragments to heal and fuse.

Sacropelvic fixation is also utilized in the treatment of scoliosis and adult scoliotic deformities. Pelvic fixation is advantageous in providing long fusions and improved fusion rates.

However, current pelvic fixation systems involve the use of multiple pelvic screws, pedicle screws, plates, hooks, and rods across the lumbosacral pelvis. Further, risk factors associated with pseudarthrosis may adversely impact achieving a biomechanical stable and robust distal fixation.

While a variety of pelvic stabilization techniques are employed, providing a biomechanically strong fixation for unstable pelvic fractures remains a challenge.

Current pelvic stabilization techniques include the use of external fixators or plate fixators close to the pelvic ring. Primary measures for pelvic fixation include the use of a pelvic harness or a pelvic c-clamp. The procedure using external fixators generally includes one pin extending from one pelvic bone and the other pin extending from the next pelvis bone, and both pins are connected using a connector external to the patient body.

While external fixators may provide primary stability, the technique is not useful to achieve complete patient mobilization. Further, treating severe pelvic injuries may require prolonged intensive care and the placement of external fixators may interfere with post-operative care.

Commonly used external fixators involve external plating or plate fixator arrangements to stabilize the pelvis. The external plates often require performance of invasive procedures. Potential disadvantages associated with the use of external fixators include mobility restrictions, prolonged immobilizations, surgical site infections, ongoing pain, limitation of fixation points, distal implant pullout, failure of fixation, loosening of fixator, and screw pullout. Moreover, external fixators have limitations in use for the stabilization of open fractures or fractures with extensive soft tissue contusion. Further, external fixators may be associated with long healing and fusion times, pin-track infection, and the risk of septic complications when secondary plating or screws are performed.

Lesions of the urogenital and digestive systems are common in high-impact pelvic injuries. These associated lesions are treated through surgical treatments. In a few cases, repeated surgical interventions are necessary. The use of external fixators or spinal implants directly restricts the operative field while increasing the risk of pin tract infections.

Internal fixation systems are also used. These systems commonly violate the sacroiliac joint by insertion of screws into the sacroiliac joint to create a stabilization column. This increases the risk of nerve damage, deep tissue damage, mobility restrictions, inflammation, pain, and infection at the insertion site.

The iliosacral screw fixation system involves a partially threaded lag screw fluoroscopically guided into the sacroiliac joint for the treatment of sacroiliac dislocation and sacral fractures. Risks associated with iliosacral screw fixation include fixation failure in cases of unstable pelvic injuries, damage of upper sacral nerve root tunnels, and limitation of use in cases of sacral deformities such as the dysmorphic pelvis. The iliosacral screw is inserted parallel to the sacroiliac joint, and the procedure may not be available to patients with abnormal anatomy.

Moreover, a transsacral screw may be required to supplement the standard sacroiliac joint screw. In some cases, an external fixator may be required to correct internal rotation prior to the iliosacral screw insertion. Further, the sacrum bone may be damaged due to the high energy impact injuring the pelvis. Therefore, the sacrum bone may not be available to accept the sacral screw Moreover, a few known techniques of internal fixation require extensive surgical exposure to the deep structures of the pelvis. Problems associated with extensive surgical exposure include delay in wound healing, damage to major nerves and tissues, and increased risk of infection.

Yet another technique for pelvic stabilization includes percutaneously closing and stabilizing the pelvic ring using spinal implants. This technique increases exposure levels, and risk of infections, and may cause interference during the performance of abdominal follow-up procedures.

Traumatic pelvic instability requires a fixator with improved biomechanical stabilization to maximize the callus formation and fusion of broken bone fragments. Posterior pelvic fixation may be inadequate to stabilize the pelvis in isolation. Therefore, stabilization means may be used including plates, medullary screws, or external fixation. However, the external fixation constructs are associated with various drawbacks including high infection rates of the transcutaneous pins, impaired patient mobilization, and insufficient biomechanical strength of the fixator.

Accordingly, there is a need for an improved system and method for pelvic stabilization that overcomes at least some of the disadvantages of existing systems and methods.

### SUMMARY

A pelvic fixation device is provided including a first fixation member pair, a second fixation member pair, and a connecting rod. The first fixation member pair includes a first main member and a first interlocking member. The first main member is defined with a proximal end comprising a connector to obliquely receive the first interlocking member to prevent rotation. The second fixation member pair includes a second main member and a second interlocking member. The second main member is defined with a second proximal end comprising a second connector to obliquely receive the second interlocking member to prevent rotation. The connecting rod is defined with two ends to securely connect with the proximal end of the first fixation member pair at one end of the connecting rod, and the second proximal end of the second fixation member pair at other end of the connecting rod, thereby laterally stabilizing each half of the pelvis.

The connecting rod of the pelvic fixation device may be defined as having curved or straight configurations.

The connecting rod of the pelvic fixation device may bypass the sacroiliac joint.

The connecting rod of the pelvic fixation device passes posterior to the sacral spinal lamina.

The connecting rod of the pelvic fixation device may be attached to the proximal end of the first main member and the second proximal end of the second main member.

The first main member and the second main member of the pelvic fixation device may be tapered in response to thickness of a receiving bone.

The first interlocking member and the second interlocking member of the pelvic fixation device may be defined as set screws extending from the proximal end of the first main member and the second proximal end of the second main member respectively.

The first interlocking member and the second interlocking member of the pelvic fixation device may be defined as threaded screws.

The threaded screws may be partially threaded or fully threaded.

In an embodiment, the first main member and the second main member of the pelvic fixation device may define proximate diameter of 12 millimetres at the proximal end and proximate diameter of 6 millimetres at a distal end. The first main member and the second main member of the pelvic fixation device may each define proximate length of between 80 millimetres to 100 millimetres. The first interlocking member and the second interlocking member of the pelvic fixation device may each define proximate length of between 50 millimetres to 70 millimetres. The connecting rod of the pelvic fixation device may define proximate length of between 100 millimetres to 140 millimetres. The connecting rod of the pelvic fixation device may define proximate diameter of between 5.5 millimetres to 7.5 millimetres. A person of skill would appreciate that the dimensions of the device depends on the anatomy of the subject being treated.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a schematic diagram of a human pelvis.

FIG. 2 is an unassembled perspective view of a pelvic fixation device, in accordance with an embodiment.

FIG. 3 is an assembled perspective view of the pelvic fixation device of FIG. 2.

FIG. 4 is a modified pedicle screw of the pelvic fixation device, in accordance with an embodiment.

FIG. 5A is a frontal view of a screw pair of a pelvic fixation device in an assembled configuration in the pelvic bone, according to an embodiment.

FIG. 5B is a frontal view of a screw pair and a connecting rod of the pelvic fixation device in the assembled configuration in the pelvic bone, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

Further, although process steps, method steps, algorithms or the like may be described (in the disclosure and/or in the claims) in a sequential order, such processes, methods and algorithms may be configured to work in alternate orders. In other words, any sequence or order of steps that may be described does not necessarily indicate a requirement that the steps be performed in that order. The steps of processes described herein may be performed in any order that is practical. Further, some steps may be performed simultaneously.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

Percutaneous posterior fixation is used for stabilization of the disrupted pelvis resulting from trauma or a medical condition. Since the main stability of the pelvis is sustained by the posterior ring, posterior fixation is advantageous in rapidly stabilizing the pelvis after a fracture.

The following relates generally to a pelvic fixation device, and more particularly to a posterior pelvic fixation device for fixing and stabilizing the pelvis as part of medical treatment.

The posterior pelvic fixation device described herein allows a minimally invasive process leading to less operation time due to small incisions and reduction of blood loss compared to current fixation devices such as plates used in external fixation. Further, the posterior pelvic fixation device provides for a robust and bio-mechanically stronger fixation while avoiding infixing a device component into the sacro-iliac joints.

The device is a system of interlockable fixation members linked by a connecting member that is useful for percutaneous posterior fixation of the pelvis for injuries or conditions that cause pelvis instability, such as hypermobility. Two fixation member pairs are used, one for each iliac wing to capture the iliac wing and then a stout connecting rod links the two pairs of fixaton members thereby connecting each half of the pelvis from one iliac wing to another iliac wing. The device is configured such that the stout connecting rod avoids infixing into the sacro-iliac joints. Instead, it passes posterior to the sacral spinal lamina.

Each pair of fixation members can include a main screw and an interlocking screw. The main screw comprises an elongated shaft that includes a proximal section and a distal section. The proximal section comprises an interlocking hole and a connecting hole. The interlocking hole is configured to receive the interlocking screw, which thereby extends through the main screw. Alternative to an interlocking hole can be an interlocking slot that alllos for the angel between the main screw and the interlocking screw to be adjusted along a plane parallel with the axis of the main screw shaft. The interlocking screw comprises an elongated shaft that is smaller in diameter compared to the main screw. The use of an interlocking screw prevents the rotational motion of the main screw when the two screws are anchored into bone. The connecting hole is configured to receive one end of the connecting rod, which in situ will provide lateral compression to the pelvis. Since a screw is inserted in the iliac on each side of the sacrum and the connecting rod bypasses the sacroiliac joint, the present device may be used in lieu of a sacroiliac screw and anterior plating/external fixation that is used today. However, the device described herein does not preclude placement of other devices such as sacro-iliac screws or anterior plates that may be deemed necessary in the event of a severe pelvic disruption.

The pelvic fixation device described herein provides for the restoration of ventral stability by applying a biomechanically robust internal fixation. The device may be used on patients affected by pelvic injuries, pelvic ring fractures, or pelvic ring injuries with transsymphysial instability. Following a minimally invasive approach without violation of the sacroiliac joint, the device provides for less soft tissue damage compared to external plating or fixation. The stability of the device is improved with the use of screw pairs, each including a main screw and an interlocking screw connected at the proximal section of the main screw. The use of an interlocking screw prevents the rotational motion of the main screw during use.

The present device may also be used for connecting with other spine fixation devices for surgical management of lumbo-pelvic disruptions and as pelvic fixation for lumbo-pelvic fixation commonly used for scoliosis surgery.

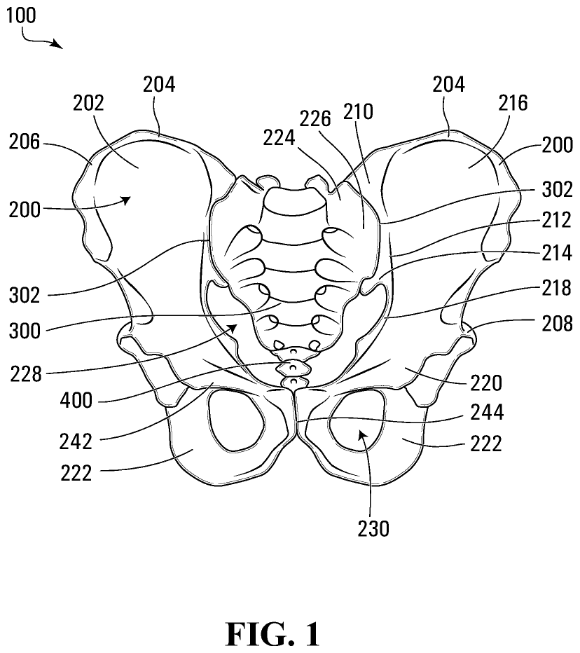

Referring now to FIG. 1, shown therein is a schematic diagram of a human pelvis 100.

Pelvis 100 includes the area between the abdomen and the lower extremities. The skeletal structure of the pelvis supports the contents of the abdomen and assists in transferring weight from the spine to the lower limbs. The skeletal structure of the pelvis includes the two hip bones 200, the sacrum 300, and the coccyx 400 attached inferiorly to the sacrum. The bones of the pelvis are strongly connected to each other via ligaments to create a mostly immobile and weight-bearing structure. The structure provides for the transfer of body weight laterally from the vertebral column, through the pelvic girdle and the hip joints into the lower limbs. Reconstruction and stabilization of the pelvic structure, therefore, becomes critical in the treatment of a pelvic injury.

The hip bone 200 is formed as large, curved bones creating lateral and anterior aspects of the pelvis. The hip bone 200 includes three key regions ilium 202, ischium 222, and pubis 242.

The ilium 202 is formed as a fan-like, superior region creating the largest part of the hip bone. The ilium 202 is firmly united to the sacrum 300 at the largely immobile sacroiliac joint 302. The ischium 222 includes the posteroinferior region of each hip bone. The pubis 242 includes the anterior portion of the hip bone. The pubis curves medially, where it joins to the pubis of the opposite hip bone at a specialized joint called the pubic symphysis 244.

The ilium 202 includes a curved, superior margin called the iliac crest 204. The iliac crest 204 includes a rounded, anterior termination called the anterior superior iliac spine 206. An anterior inferior iliac spine 208 is a rounded protuberance located inferior to the anterior superior iliac spine. The superior iliac spine 206 and the anterior inferior iliac spine 208 serve as attachment points for muscles of the thigh. At the back, the iliac crest 204 curves downward to terminate as the posterior superior iliac spine 210. The posterior superior iliac spine 210 may be identified as a depression or a “dimple” located on the lower back. The posterior superior iliac spine 210 is further connected to the sacral spinal canal in the sacrum 300. The sacral spinal canal includes the sacral spinal lamina. Posterior inferior iliac spine 212 is located at the inferior end of a large, roughened area called the auricular surface 214 of the ilium 202. The auricular surface articulates with the auricular surface of the sacrum to form the sacroiliac joint. Both the posterior superior and posterior inferior iliac spines serve as attachment points for the muscles and very strong ligaments that support the sacroiliac joint.

Iliac fossa 216 is the shallow depression located on the anteromedial (internal) surface of the upper ilium. The inferior margin of the iliac fossa 216 is formed by the arcuate line of the ilium. The arcuate line is formed as a ridge occurring due to the pronounced change in curvature between the upper and lower portions of the ilium. Greater sciatic notch 218 is the large, inverted U-shaped indentation located on the posterior margin of the lower ilium.

The three areas of each hip bone 200 including the ilium 202, ischium 222, and the pubis 242, converge centrally to form a deep, cup-shaped cavity called the acetabulum 220. The acetabulum 220 is located on the lateral side of the hip bone and is part of the hip joint.

The largely immobile sacroiliac joint 302 in the pelvis 100 is supported by strong ligaments that are attached between the sacrum 300 and ilium portions 202 of the hip bone. The ligaments include the anterior sacroiliac ligament 224 on the anterior side of the joint and the posterior sacroiliac ligament 226 on the posterior side. Two additional ligaments span across the sacrum and hip bone including the sacrospinous ligament 228 running from the sacrum to the ischial spine, and the sacrotuberous ligament 230 running from the sacrum to the ischial tuberosity.

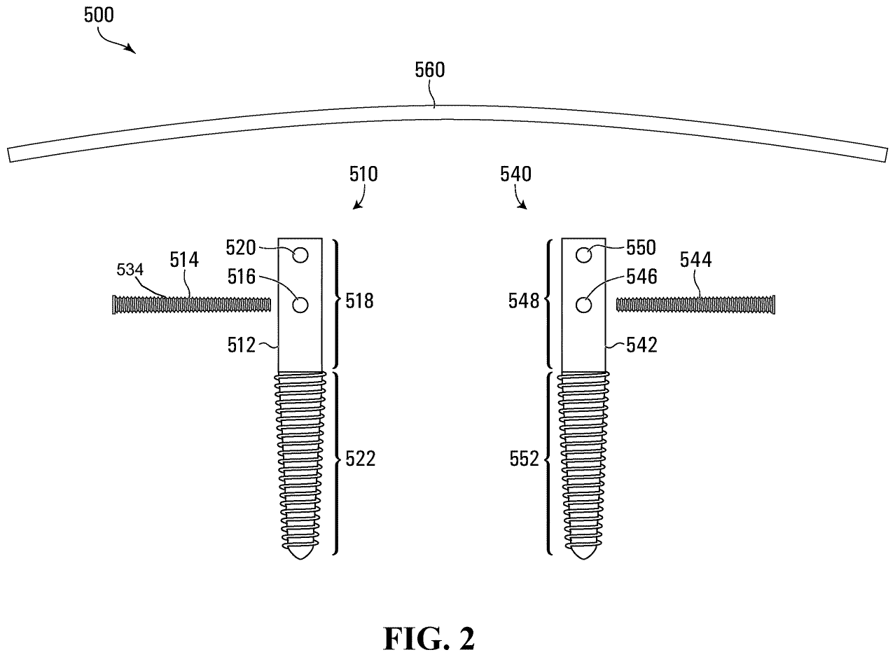

Referring now to FIG. 2, shown therein is an unassembled perspective view of an embodiment of the posterior pelvic fixation device 500 in accordance with the present disclosure.

The posterior pelvic fixation device includes two pairs 510, 540 of interlocking screws; one screw pair 510, 540 fixed to each of the iliums 202 (FIG. 1) on the posterior side thereof and a connecting rod 560 connecting, in a rigid or substantially rigid manner, the interlocked screw pairs 510, 540 without infixing into the sacroiliac joint. The two interlocked screw pairs comprise of the first screw pair 510 and the second screw pair 540. The first screw pair include first main screw 512 and the first interlocking screw 514. The second screw pair includes the second main screw 542 and the second interlocking screw 544. The main screw is thicker and longer compared to the respective interlocking screw.

The first main screw 512 includes an interlocking hole 516 in the proximal section 518 of the screw 512 that is configured to receive the first interlocking screw 514, thereby resisting rotational motion of the main screw and interlocking screw when the main screw and the interlocking screw are anchored at the distal ends. The interlocking hole 516 and interlocking screw 514 can also be configured to secure the interlocking screw, thereby resisting translational and rotational motion of the interlocking screw. The first main screw 512 further includes a connecting hole 520 between the interlocking hole 516 and the screw's proximal end to receive and secure the connecting rod 560, thereby resisting translational and rotational motion of the connecting rod 560. In an alternative embodiment (not shown), the interlocking hole may be between the connecting hole 520 and the screw's proximal end. The first main screw and/or the first insterlocking screw are configured such that 95% to 100%, preferably 98-100%, of the length thereof can be embedded in the ilium, such as the iliac spine.

Similarly, the second main screw 542 includes an interlocking hole 546 at in the proximal section 548 of the screw 512 that is configured to receive the second interlocking screw 544, thereby resisting rotational motion of the main screw and interlocking screw when the main screw and the interlocking screw are anchored at the distal ends. The interlocking hole 546 and interlocking screw 544 can also be configured to receive and secure the interlocking screw, thereby resisting translational and rotational motion of the interlocking screw. The second main screw 542 further includes a connecting hole 550 between the interlocking hole 546 and the screw's proximal end configured to receive and secure the connecting rod 560, thereby resisting translational and rotational motion of the connecting rod 560. In an alternative embodiment (not shown), the interlocking hole may be between the connecting hole and the screw's proximal end. The second main screw and/or the second interlocking screw are configured such that 95% to 100%, preferably 98-100%, of the length thereof can be embedded in the ilium, such as the iliac spine.

Thus, in total, for the embodiment shown, four screws are employed in the pelvic fixation device.

The main screws 512, 542, to facilitate penetration of the iliac bone, can taper within the distal section 522, 552 toward the distal end such that the narrowest diameter of the distal section 522, 552 is at the distal end of the screw. In the embodiment shown, the proximal section 518, 548 does not taper but has a constant diameter along its length.

Similarly, the interlocking screws 514, 544, to facilitate insertion into the interlocking hole and penetration of the iliac bone, can comprise a taper to its distal end, whereby the distal end has the narrowest diameter.

Once inserted, the proximal section of the interlocking screw 514, 544 couples to the proximal section of the main screw 512, 542. The coupling of the two components can be via a mated fit, i.e., the interlocking hole diameter is slightly larger than the proximal section of the interlocking screw such that the inner surface of the interlocking hole receives the interlocking screw and contacts the surface of interlocking screw at multiple points along the circumference of the screw. In the embodiment shown in FIG. 2, the interlocking screws 514, 544 couples via threads 534 on the interlocking screw deforming the inner wall of the interlocking hole of the main screw 512, 542 as a tapering interlocking screw is screwed into the hole, thereby resisting translational motion. The reverse of this configuration is also possible where the threads are on the inner wall of the interlocking hole and deform the surface of the interlocking screw. Alternatively, the main screw is configured to couple with the interlocking screw via threads 534 on the interlocking screw and threads on the inner wall of the interlocking hole, thereby resisting translational motion along the axis of the interlocking screw. A person of skill would appreciate that other reversible or irreversible connecting mechanisms could be used.

The main screws 512, 542 and the interlocking screws 514, 544 are configured to anchor to bone preferably in a reversible manner, with the distal sections thereof. In the embodiment shown, the main screws 512, 542 and the interlocking screws 514, 544 are threaded at the sections to be inserted into the pelvic bone and the threads are configured to penetrate and securely couple with the bone.

As described above, the main screws 512, 542 and the interlocking screws 514, 544 are configured to couple to each other in the proximal section thereof. The interlocking screws 514, 544, also at the section to contact the main screw can be threaded and configured to securely couple to the main screw. Accordingly, the main screws 512, 542 and interlocking screws 514, 544 may be partially threaded or fully threaded.

The dimensions of the main screws 512, 542, the interlocking screws 514, 544, and the connecting rod 560 may be determined based on the incident bone thickness, the pelvic size of the subject to receive the device, and the tensile strength material of which the device is fabricated. The thickness of the main screws 512, 542 and the interlocking screws 514, 544 may also depend upon the bone mineral density (BMD). X-ray or medical imaging may be performed to identify any bone abnormalities or defects before determining the screw trajectory.

Preferable material of construction for main screws 512, 542, interlocking screws 514, 544, and connecting rod 560 may include cobalt or titanium alloys.

Preferable length and diameter is such that the main screws 512, 542 and the interlocking screws 514, 544 remain within the inner and outer tables of the ilium when the main and interlocking screws intersect at an angle of approximately 90°.

The length of the main screws 512, 542 and the interlocking screws 514, 544 may be determined based on the pre-operative imaging and intra-operative probing. Length of the main screws 512, 542 and the interlocking screws 514, 544 may also depend on the tensile strength of the screws to prevent any breakage when inserted in the pelvis.

Preferably, the length of the main screws 512, 542 is greater than the length of the interlocking screws 514.

The main screw can have a proximal section with a greater diameter than the diameter of the distal section. A maximum diameter of the distal section of main screws 512, 542 that penetrates the bone can be the same as the proximal section with the distal section tapering by 70% to 50% of the maximum diameter at the distal end of the main screws. By way of example, for an adult subject, the proximal sections 518, 548 of the main screws 512, 542 can have a thicker diameter of 10 mm to 12 mm to accommodate the corresponding interlocking screws 514, 544 and the connecting rod 560. The maximum diameter of the distal section can be 8 mm to 12 mm and taper to the distal end diameter of the distal sections 522, 542 of the main screws 512, 542 to 4 mm to 6 mm.

By way of example, for an adult subject, the length of the main screws 512, 542 may be between 80 millimetres to 100 millimetres.

By way of example, for an adult subject, the maximum diameter of the interlocking screws 514, 544 may be approximately 6 mm. The diameter of the interlocking screws 514, 544 can be constant along the entire length of the screw, or the interlocking screw can taper to 50 to 80% of the maximum diameter at the distal, bone penetrating end of the screws 514, 544.

By way of example, for an adult subject, the length of the interlocking screws 514, 544 may be between 50 millimetres to 70 millimetres.

The length of the connecting rod 560 is sufficient to span the distance between the connecting holes of the two main screws, when the main screws are anchored into the ilium. By way of example, for an adult subject, the length of the connecting rod 560 may be approximately 100 to 200 mm, or any length corresponding to the length between the iliac wings. The connecting rod 560 can be straight or have up to 40° of curvature or up to about 20° to 30° of curvature.

The connecting rod is configured to be rigid or sufficiently rigid in order to provide the stability to the sacroiliac joint. The diameter of the rod can depend on the degree of bendability of the material being used at various diameters. By way of example, a diameter of the connecting rod 560 that is a cobalt or titanium alloy may be between 2 to 8 millimetres, such as 3 millimetres to 7 millimetres or 5.5 to 7.5 millimetres.

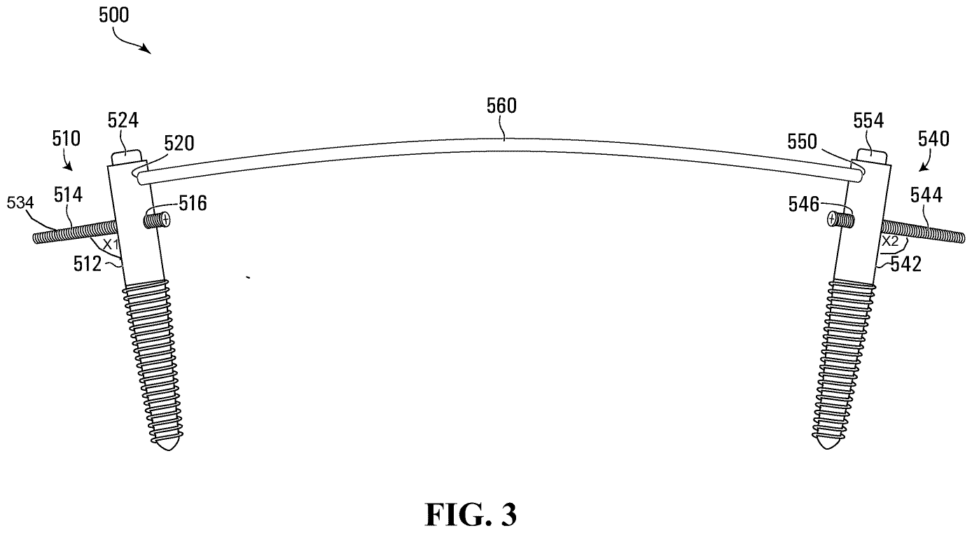

Referring now to FIG. 3, shown therein is an assembled perspective view of the posterior pelvic fixation device 500 depicted in FIG. 2 and described above. In particular, FIG. 3 depicts fixation device 500 with the main screws 512, 542 receiving the interlocking screw 514, 544, respectively, through the interlocking hole 516, 546. The main screws 512, 542 and the interlocking screw 514, 544, when passing through the respective interlocking hole 516, 546, each form respective internal angle x1, x2. The interlocking screw by being also fixed to the ilium prevents rotation of the main screw 512, thereby strengthening the fixation of the interlocking screw pair to the ilium. The angle between the interlocking screw and the main screw can vary based on the anatomy of the subject. FIG. 3 depicts x1 and x2 as being approximately 90 degrees, yet the internal angle x1 and x2 can be between 30 to 90 degrees, preferably, the internal angle x1 and internal angle x2 are between 30 degrees to 70 degrees or 40 to 60 degrees.

Also as depicted in FIG. 3, one end 562 of the connecting rod 560 is inserted in the first connecting hole 520 within the proximal section 518 of the first main screw 512 between the interlocking hole 516 and the proximal end of the main screw 512. The other end 564 of the connecting rod 560 is inserted in the second connecting hole 550 within the proximal section 548 of the second main screw 542 between the interlocking hole 546 and the proximal end of the main screw 542.

The first main screw 512 and the second main screw 542 can be configured to receive a locking caps 524, 554. The cap 524, 554 is configured to press fit into the proximal end opening of the main screw 512, 544 to secure the connecting rod 560 when the rod is inserted in the connecting hole 520 and connecting hole 550 at each end. Alternative to the caps 524, 554, the proximal end of the main screw may be configured to receive a set screw to lock the connecting rod. The set screw and the interior wall of the main screw defining a proximal end opening thereof can be threaded and sized so that the set screw screws into the proximal end opening. Tightening the set screw will lock the connecting rod relative to the main screw.

Preferably, the connecting rod 560 is inserted through the connecting hole 520 the proximal section 518 of the first main screw 512 in the first screw pair 510. The connecting rod 560 is then advanced towards the second screw pair 540. The other end 564 of the connecting rod 560 is inserted into the connecting hole 550 of the second main screw 542. When the desired position of the connecting rod 560 is achieved, the rod may be secured by pressing the locking caps 524 and 554 respectively into both end, thereby fixing the relative position of the connecting rod 560 and the main screws 512, 544.

The connecting rod 560 depicted in FIG. 2 does not comprise threads at the ends to engage with the connecting hole, thus relying solely on the locking caps or a set screw to lock its position. However, in an alternative embodiment, thread on the ends 562 and 564 of the connecting rod 560 may be provided to aid in fixing the position of the connecting rod relative to the main screw.

The connecting rod 560 can be curved according to the shape of the posterior ring and bypass the sacral spinal canal. Further, the connecting rod 560 passes posterior to the sacral spinal lamina. Bending tools may be used to curve the rod to the desired curvature. Further, a pre-curved connecting rod may be used. The pre-curved connecting rod is configured to pass posteriorly to the sacral lamina safely.

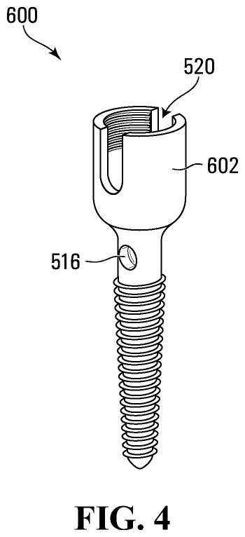

Referring now to FIG. 4, shown therein is an alternative embodiment of a main screw that can be used as the first and second main screws of the pelvic fixation device 500 described above. This alternative main screw 600 of FIG. 4 is essentially a pedicle screw adapted to receive the interlocking screw and the connecting rod. As shown, main screw 600 is configured to receive and lock in position the ends 562 and 564 of the connecting rod 560. The main screw 600 comprises an open proximal end 602 for top loading the connection rod. The screw 600 comprises opposite facing connecting slots open at the proximal end 602 of the main screw and configured such that the connecting rod 560 extends through both slots. The main screw 600 has a threaded inner wall defining the open proximal end configured to receive a set screw that can lock the position of the connecting rod. For a top loadable embedment, in implantation, a channel could be created in the bone adjacent the slot such that the connecting rod may extend therein to thereby extend through the slot to be locked in position.

Another embodiment of a main screw not shown could comprise a proximal section comprising the connecting hole that is configured to rotate relative to the distal section of the main screw. The distal section being the same as that described above. The distal section being configured to penetrate and anchor to bone. The interlocking hole would be located adjacent the rotatable portion of the main screw such that the angle of the main screw and the interlocking screw is fixed but the angle of the connecting rod and the main screw can be adjusted. The main screw is configured such that the rotatable proximal section thereof would be locked in position when a locking cap or set screw is inserted into a proximal opening to lock the position of the connecting rod.

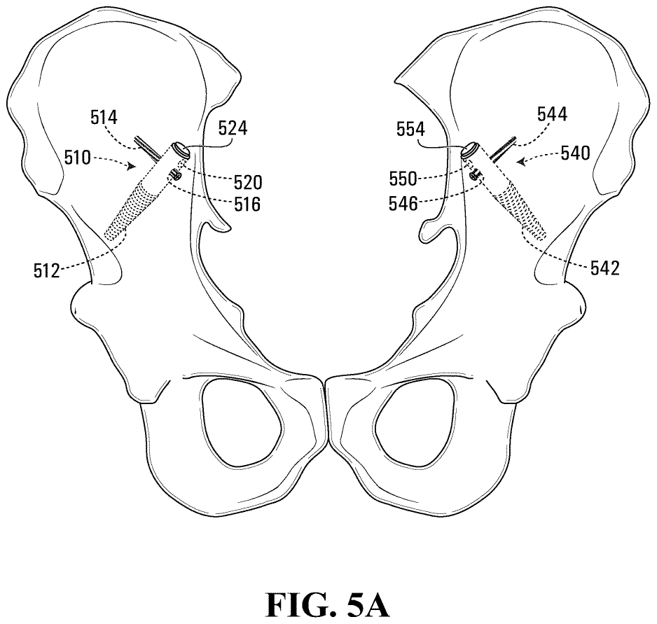

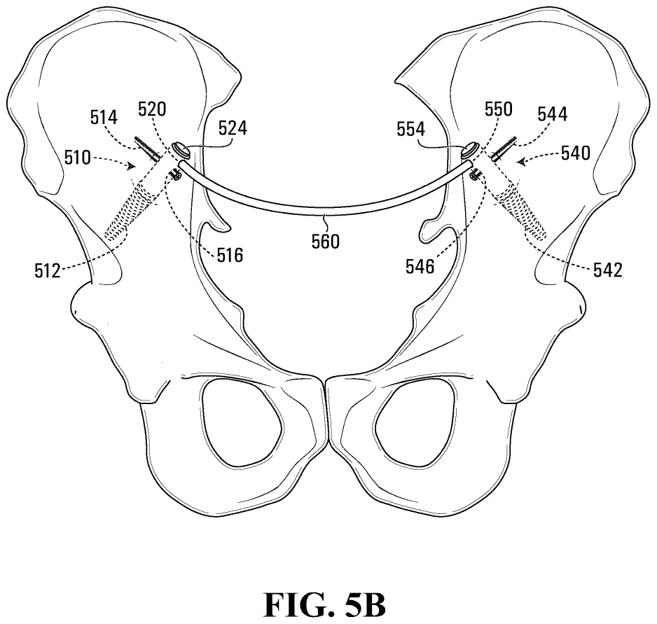

Referring now to FIG. 5B, shown therein is the pelvic fixation device 500 of FIGS. 2 and 3 and described above in an assembled configuration and fixed to the right and left ilium. FIG. 5A is the same as FIG. 5B but depicted without the connecting rod 560. It is noted that FIGS. 5A and 5B are not drawn to scale, so the size of the iliac wings relative to the device is not to scale.

As shown, the first main screw 512 and the second main screw 542 are inserted in the posterior superior iliac crest 210 opposite from the other. The insertion in the posterior superior iliac crest 210 provides a longer insertion channel and a biomechanically stronger placement of the larger main screw. The placement of the first main screw 512 and the second main screw 542 in either of the posterior superior iliac crests 210 may be facilitated with the use of intra-operative imaging techniques and computer-assisted tomographic image guidance to guide the screw trajectory. Further, radiography or 3D or CT imaging may be used to guide screw placement. The screws may be inserted with X-ray or CT based navigation, or with a surgical robot. Alternatively, anatomical and bony landmarks may be used for the insertion of the main screw into the posterior superior iliac crest.

According to an embodiment, once the incident point is identified for insertion of the first main screw 512 and the second main screw 542, subperiosteal dissection is made to expose the incident posterior superior iliac crest 210. Pedicle probes or pedicle finders may be used to determine the trajectory of the screws.

Alternatively, a guidewire may be used for drilling into the planned first main screw 512 and the second main screw 542. Preferably, with the adequate placement of a first main wire verified by intra-operative imaging, the hole for the screw may be prepared with cannulated drills and taps. Following this preparation, the first main screw 512 may be inserted and then preparations may be made for the second main screw 542. A guiding arm attached to the first main screw 512 may guide the guidewire through bone, and then subsequently through the appropriate hole in the first main screw 512. The hole for the second main screw 542 may then be prepared with cannulated drills and taps and then inserted.

The track created in each iliac wing using the pedicle probes may form the path for the first main screw 512 and the second main screw 542. The main screw may be inserted from the posterior superior iliac crest 210 and directed towards the anterior inferior iliac spine 208. The main screws may pass through the cortical bone and end at the region in the ileum above the sciatic notch 218. This provides a thick column of bone to receive the main screw and creates a robust insertion.

The first main screw 512 and the second main screw 542 may use an alternative trajectory for main screw placement according to special circumstances. These include the location of the fracture, the presence of unusual pelvic morphology, or the identification of a more suitable region in the ilium to receive the main screw. The alternative trajectory is preferably selected to prevent the risk of acetabular violation, cortical breach, or damage to neural and vascular structures. The first main screw 512 and the second main screw 542 preferably remains within the inner and outer tables of the ilium. The first main screw 512 and the second main screw 542 trajectories preferably prevent any violation of the hip joint or the sacro-iliac joint.

According to an embodiment, only the distal sections 522, 552 of the first main screw 512 and the second main screw 542 respectively are inserted into the iliac wing. In the embodiment shown, the distal sections 522, 552 are threaded to facilitate the fixation to the pelvic bone. Other mechanisms for fixing an elongated support in a bone may also be used. Such mechanisms are typically desired be reversible as the device is intended for removal once the subject's injury is healed or otherwise stabilized. When the first main screw 512 and the second main screw 542 are inserted, the proximal sections 518, 548 of the first main screw 512 and the second main screw 542, may preferentially remain completely within the bone.

Once the first main screw 512 and the second main screw 542 are placed on either side of the iliac wing, the corresponding interlocking screws 514, 544 are inserted through the interlocking holes 516, 546 in each of the main screws 512, 542. The first interlocking screw 514 is inserted through the interlocking hole 516 in the first main screw 512 to create the first screw pair arrangement 510. The second interlocking screw 544 is inserted through the interlocking hole 546 in the second main screw 542 to create the second screw pair arrangement 540. FIG. 5A illustrates the first screw pair 510 and the second screw pair 540 inserted in the ilium before the connecting rod 560 is placed.

To facilitate the insertion of the first interlocking screw 514 and the second interlocking screw 516 in the respective first main screw 512 and the second main screw 542, guiding tools similar to those used for the insertion of the main screws may be employed. Guiding tools include intraoperative fluoroscopy or intraoperative 3D or CT imaging, computer image guidance, or surgical robotic devices. Alternatively, anatomical and bony landmarks may be used for the insertion of the large screw into the posterior superior iliac crest.

Once the trajectory for the first interlocking screw 514 and the second interlocking screw 516 is determined, the interlocking screws are inserted through the respective interlocking holes 516, 546 into the iliac wing. In an embodiment, the head of both screws may be buried just below the cortical margin of the bone to avoid having prominent hardware post operatively. Each screw may be inserted through a separate small incision.

According to an embodiment, interlocking holes 516, 546 on the proximal section of the respective first main screw 512 and the second main screw 542 are identified for insertion of the interlocking screws 514, 544. An aiming device may be used to locate the interlocking holes 516, 546 on the main screws 512, 542. The interlocking screws 514, 544 are then inserted through the interlocking holes 516, 546 and into the ilium. The screws may be inserted through a separate incision and inserted until the head is located just below the cortical bone.

The interlocking hole of the main screw may be lined up by screw rotation to align the interlocking hole with the inner table of the ilium, into which the interlocking screw is inserted. The interlocking screws may then be inserted with guiding arms attached to the main screw that ensure its trajectory through the interlocking hole. Preferably, the inner angle (x1, x2) between the main screws 512, 542 and the corresponding interlocking screws 514, 544 is established to prevent rotation of the main screws 512, 542 post-insertion and improve the construct's stability. Advantageously, the use of interlocked screw pairs 510, 540 including main screws 512, 542 and corresponding interlocking screws 514, 544 on either side of the pelvis provides for biomechanically stable fixation. The interlocking screws 514, 544 provide an angular stable locking feature while reducing interfragmentary movements in case of a fracture.

Preferably, pedicle probes may be used to create an angular path for the interlocking screws 514, 544. The interlocking screws 514, 544 are inserted at an angular plane different from the main screws 512, 542. The trajectory of the interlocking screw may be determined to achieve internal angular stable locking, preferably without the need for external fixators.

The interlocking screws 514, 544 may use an alternative trajectory at an angle from the trajectory of the main screws 512, 542 as per applicable circumstances. The angular trajectory of interlocking screws is provided to prevent rotation of the main screw. Applicable circumstances relevant to the trajectory determination for the interlocking screws 514, 544 also include the location of the fracture, the presence of unusual pelvic morphology, or the identification of a more suitable region in the ilium to receive the main screw. The alternative trajectory is preferably selected to prevent the risk of acetabular violation, cortical breach, or damage to neural and vascular structures. The interlocking screws 514, 544 preferably remain within the inner and outer tables of the ilium. The interlocking screw trajectory preferably prevents any violation of the hip joint.

Proximal locking is achieved by placement of the interlocking screws 514, 544 through interlocking holes 516, 546 at the proximal section of the main screw. Angularly stable and biomechanically stronger anti-rotational fixation between the main screws 512, 542 and the corresponding interlocking screws 514, 544 is achieved once the screw pair is inserted in the iliac wing.

On placement of the first screw pair 510 and the second screw pair 540 on either side of the pelvic wings, a connecting rod 560 is subcutaneously inserted and locked into position to fix relative position of the screw pairs 510, 540, thereby stabilizing sacroiliac joint and the sacrum and ilium interfacing with said joint. According to an embodiment, one end 562 of the connecting rod 560 is inserted in the first connecting hole 520 within the proximal section 518 of the first main screw 512. The connecting rod 520 is then advanced towards the second screw pair 540. The other end 564 of the connecting rod 560 is inserted in the second connecting hole 550 within the proximal section 548 of the second main screw 542. In an embodiment, a flexible test rod may be used to define the desired length of the connecting rod before inserting the connecting rod 560 itself.

The connecting rod 560 may be curved according to the shape of the posterior ring and bypass the sacroiliac joint. Further, the connecting rod may pass posterior to the sacral spinal lamina. Bending tools may be used to curve the rod to the desired curvature. In an embodiment, the test rod may also be used to determine the desired degree of curvature of the connecting rod 560. Alternatively, a pre-curved connecting rod may be used. The pre-curved connecting rod is configured to pass posterior to the sacral lamina safely. FIG. 5B illustrates the pelvic fixation device 100 in the assembled configuration in the pelvic bone with the connecting rod 560 extending between and securely coupled to the first screw pair 510 and the second screw pair 540.

When the desired position of the connecting rod 560 is achieved, the connecting rod 560 is locked into position relative to the mains screws. The cap 524, 554 (or alternatively, a set screw) can be used to lock the connecting rod after the rod is inserted in the connecting hole 520 and connecting hole 550 at each end and properly positioned. The connecting rod 560 inserted into the pelvic bone and coupled to the main screw thereby provides longitudinal alignment to the screw pairs 510, 540.

As shown in FIGS. 5A and 5B, the proximal sections 518, 548 of the main screws 512, 542 are embedded within the bone. Accordingly, tunnels may be created in the bone for the connecting rod 560 to access the proximal sections 518, 548 of the main screws 512 and 542, in particular, the connecting hole, and to receive the connecting rod.

The first screw pair 510 comprising the first main screw 512 and the corresponding first interlocking screw 514 secured to the proximal section 518 of the first main screw 512. The first main screw 512 and the first interlocking screw 514 are inserted in one of the iliac wings. The first main screw 512 and the first interlocking screw 514 are aligned at an angle x1 to each other to impede rotation of the first main screw 512 and thereby improve construct stability.

The second screw pair 540 refers to the second main screw 542 and the corresponding second interlocking screw 544 connected to the proximal end 548 of the second main screw 542. The second main screw 542 and the second interlocking screw 544 are inserted in the other iliac wing. The second main screw 542 and the second interlocking screw 544 are aligned at an angle x2 to each other to impede rotation of the second main screw 542 and thereby improve construct stability.

According to an embodiment, one end 562 of the connecting rod is inserted in the connecting hole 520 at the proximal end of the proximal section 518 of the first main screw 512. The other end 564 of the connecting rod 560 is inserted in the connecting hole 550 at the proximal end of the proximal section 548 of the second main screw 542.

Guiding devices attached to the main screw may be used to prepare tunnels in the ilium for the connecting rods on either side. The connecting rod 560 is inserted through the connecting hole 520 in the proximal section 518 of the first main screw 512. The connecting rod 520 is then advanced into the subcutaneous layer towards the second screw pair 540. The other end 564 of the connecting rod 560 is inserted into the connecting hole 550 of the second main screw 542.

Once the connecting rod 560 is inserted through the connecting hole 520 of the first main screw 512, the connecting rod 560 is directed towards the contralateral iliac wing which includes the inserted second screw pair 540. When the connecting rod 560 reaches the contralateral iliac wing, it will be guided through the prepared tunnel in the ilium and through the connecting hole. The other end 564 of the connecting rod 560 may be then inserted into the connecting hole 550 located on the proximal section 548 of the second main screw 542. The connecting rod 560 may bypass the sacro-iliac joint, and therefore avoid infixing into the sacro-iliac joint. Further, the connecting rod 560 passes posterior to the sacral spinal lamina. Preferably, the positioning of the connecting rod 560 may be confirmed fluoroscopically. When the desired position of the connecting rod 560 is achieved, the rod is tightened using the locking caps 524, 554. Pelvic reduction should be obtained prior to tightening of the end caps.

The connecting rod may be curved according to the shape of the posterior ring and bypass the sacral spinal canal. Bending tools may be used to curve the rod to the desired curvature. Further, a pre-curved connecting rod may be used. The pre-curved connecting rod is configured to pass behind the sacral lamina safely.

After the opposing ends 562, 564 of the connecting rod 560 are inserted in the connecting hole 520 of the first main screw 512 and the connecting hole 550 of the second main screw 542, the locking caps 524, 544 of the main screws 512, 542 may be tightened using a screwdriver. Because the heads of the main screws are not rotatable, the rod is firmly positioned when the screw locking caps 524, 544 are tightened. Therefore, the relative micro-movement between fractured sites is preferably prevented.

Biomechanical advantages are achieved with the use of interlocking screw pairs 510, 540 and a connecting rod 560 including decreasing the impact of external forces on the fixation points. The presence of a connecting hole in the main screws 512, 542 to receive the connecting rod facilitates assembly compared to U-shaped heads in the unmodified pedicle screw.

Once positioned inside the patient, the pelvic fixation device 500 provides a stable column for bone healing and fusion. Preferably, the pelvic fixation device is left in situ for an appropriate period such as 3-4 months. On the completion of bone healing, the device may be removed using similar incisions made during implantation. Due to the simple assembly of the pelvic fixation device 500, the removal process is uncomplicated. Moreover, compared to external fixation systems, the soft tissue damage is substantially reduced during the removal of the pelvic fixation device 500.

Preferably, computer tomographic (CT) imaging may be used to identify the paths for placement of the screws and the connecting rod. Comprehensive imaging including trans-axial imaging of each site of injury may be performed to identify the displacement and areas of instability.

The present pelvic fixation device 500 provides minimally invasive stabilization tools. The device is useful for the fixation of pelvic injuries or pelvic defects including multiple fractures, comminuted fractures of the pelvic ring, coagulopathies, and pelvic fixation. Advantageously, the screw pairs 510, 540 and the connecting rod 560 are placed subcutaneously without infixing into the sacroiliac joint 302. With the internal placement of the pelvic fixation device, regular care and mobilization of the patient are facilitated. Multiple abdominal follow-up procedures may be performed without the interference of the stabilization constructs.

According to an embodiment, the pelvic fixation device may be used in scoliosis treatment and correction of adult scoliotic deformities. The pelvic fixation device (without connecting rod 560) may be used for connection of long lumbosacral fusions (for any diagnosis) as a means of pelvic fixation. Such an embodiment of the device may include a special connector that attaches through holes 520 and 550 that accepts a spinal fusion rod. Such an embodiment of the pelvic fixation device may be used to replace typical iliac wing fixation screws and sacral alar screws (which violate by drilling into the sacro-iliac joint). This may provide more secure fixation to the pelvis than previously used methods or devices.

According to an embodiment, the pelvic fixation device may be used for vertebral fixation. A long posterior construct may be used extending from the thoracic spine into the sacrum to connect with the pelvic fixation device. Further, the pelvic fixation device is capable of connecting to spine fixation constructs for surgical management of lumbopelvic disruptions. The pelvic fixation device may be used for lumbopelvic fixation commonly used for scoliosis surgery. For example, a connector inserted in holes 520 and 560 may be used to secure the spinal fusion rods to the main fixation screws.

Due to the minimally invasive procedure required to install the pelvic fixation device 500, specific advantages are achieved. These include preservation of soft tissue, obviating the need for external fixators and violation of the sacroiliac joint, increased stability, easier device removal after the bone hearing, and improvement in mobility. Unlike the use of an external fixator, the risk of infection does not increase when parallel procedures such as a simultaneous laparotomy are performed. Further, depending on the fracture, the motion segments are preserved while avoiding any trauma from infixing into the sacroiliac joint.

Advantageously, the use of an interlocking screw pair including the main screw and an interlocking screw on either side of the pelvis provides for biomechanically stable fixation. The interlocking screws 514, 544 provide an angular stable locking feature while reducing interfragmentary movements in case of a fracture. By limiting the interfragmentary movements, the callus formation is stimulated to facilitate the fusion process and reduce the healing period.

Angular stable locking is achieved through the placement of main screws 512, 542 and the corresponding interlocking screws 514, 544 traversing through the main screw at an angle.

Compared to external fixators, the pelvic fixation device provide for internal fixation arrangement and no external plating is preferably required. Moreover, the screw pair 510, 540 and the connecting rod 560 are inserted through an incision remote to the pelvic fracture thereby minimally disturbing the fracture site and improving callus formation. By providing higher biomechanical stability compared to external fixators, the healing process and bone fusion are enhanced. By reducing the use of constructs and obviating the need for external fixators, the risk of surgical site infections is reduced. Moreover, a biomechanically stronger and robust fixation is achieved at the lumbosacral junction, creating a robust long fusion.

The insertion point for the main screw may be just at or above the posterior superior iliac spine and the insertion point for the interlocking screw may be just below the posterior superior iliac spine. The angle between the two may be approximately 30-70 degrees. The interlocking screw may be guided through the interlocking hole with guiding instruments attached to the main screw.

The two interlocking screw pairs that together with the connecting rod from the device are identical in construction or mirror images of each other. Thus, when reference is made in the present application to the main screw without specifying the first or second, it is understood that the features describe apply to both. Similarly, when reference is made in the present application to the interlocking screw without specifying the first or second, it is understood that the features describe apply to both the first and the second. This also applies when describing a feature of the interlocking screw pair.

While the above description provides examples of one or more apparatus, methods, or systems, it will be appreciated that other apparatus, methods, or systems may be within the scope of the claims as interpreted by one of skill in the art. What is claimed is the systems and methods as generally and specifically described herein.

1. A pelvic fixation device comprising:
a first fixation member pair configured to anchor to a pelvic bone on a right side of the medial plane at two points, the first fixation member comprising a first main member and a first interlocking member,
the first main member having a proximal section comprising a hole therethrough configured to obliquely receive and couple to the first interlocking member and comprising a distal section configured to anchor to the pelvic bone on the right side of the medial plane and
the first interlocking member comprising a proximal section that is configured to couple to the first main member and comprising a distal section configured to anchor to the pelvic bone on the right side of the medial plane;
a second fixation member pair configured to anchor to the pelvic bone on a left side of the medial plane, the second fixation member comprising a second main member and a second interlocking member,
the second main member having a proximal section comprising a hole therethrough configured to obliquely receive and fixedly couple to the second interlocking member and comprising a distal section configured to anchor to the pelvic bone on the left side of the medial plane and
the second interlocking member comprising a proximal section that fixedly couples to the second main member and comprising a distal section configured to anchor to the pelvic bone on the left side of the medial plane;
a connecting rod configured to extend between the first and second fixation members and not to infix any bony structure of the pelvis or joint space therein, the connecting rod having two ends configured to fixedly couple with the proximal section of the first fixation member pair at one end of the connecting rod, and the second proximal section of the second fixation member pair at other end of the connecting rod, thereby fixing the relative position between the first main member and the second main member.

2. A posterior pelvic fixation device comprising:
a first fixation member pair configured to anchor to the right ilium on the posterior side thereof, the first fixation member comprising a first main member and a first interlocking member,
the first main member having a proximal section comprising a hole therethrough configured to obliquely receive and fixedly couple to the first interlocking member and comprising a distal section configured to anchor to the posterior side of right ilium and
the first interlocking member comprising a proximal section that fixedly couples to the first main member and comprising a distal section configured to anchor to the posterior side of right ilium;
a second fixation member pair configured to anchor to the left ilium on the posterior side thereof, the second fixation member comprising a second main member and a second interlocking member,
the second main member having a proximal section comprising a hole therethrough configured to obliquely receive and fixedly couple to the second interlocking member and comprising a distal section configured to anchor to the posterior side of left ilium and
the second interlocking member comprising a proximal section that fixedly couples to the second main member and comprising a distal section configured to anchor to the posterior side of left ilium;
a connecting rod configured to extend between the first and second fixation members posterior to the sacral spinal lamina, the connecting rod having two ends configured to fixedly couple with the proximal section of the first fixation member pair at one end of the connecting rod, and the second proximal section of the second fixation member pair at other end of the connecting rod, thereby fixing the relative position between the first main member and the second main member.

3. The pelvic fixation device of any one of the claims 1 to 2, wherein the first main member and the second main member comprise a connecting hole or slot in the proximal section thereof that is configured for the connecting rod to extend therethrough.

4. The pelvic fixation device of claim 1 or 3, wherein the connecting rod defines an arc extending between the two ends and optionally wherein the degree of curvature of the connecting rod is 20 to 30 degrees.

5. The pelvic fixation device of any one of the claims 1 to 4, wherein the first main member and the second main member comprised a tapered distal section whereby the narrowest diameter is at the distal end of the first main member and the second main member.

6. The pelvic fixation device of any one of the claims 1 to 5, further comprising a pair of set screws or locking caps, each configured to press against the connecting rod at each end and securely couple with the main member, thereby locking the connecting rod's position relative to the main member.

7. The pelvic fixation device of any one of the claims 1 to 6, wherein the first interlocking member and the second interlocking member are either fully or partially threaded screws.

8. The pelvic fixation device of any one of the claims 1 to 7, wherein the first interlocking member and the first main member are configured to form an internal angle of 30 to 70 degrees when fixed to the pelvic bone and wherein the second interlocking member and the second main member are configured to form an internal angle of 30 to 70 degrees when fixed to the pelvic bone.

9. The pelvic fixation device of any one of the claims 1 to 8, wherein the distal section of the first main member is 50-75% of the entire length of the first main member and the proximal section of the main member is the remainder of the length and wherein the distal section of the second main member is 50-75% of the entire length of the second main member and the proximal section of the second main member is the remainder of the length.

10. The pelvic fixation device of any one of the claims 1 to 9, wherein the connecting rod is composed entirely of a cobalt or titanium alloy or
wherein the first and second main members are composed entirely of a cobalt or titanium alloy or
wherein the first and second interlocking members are composed entirely of a cobalt or titanium alloy.

11. The pelvic fixation device of any one of the claims 1 to 10, wherein the first main member and the second main member is configured such that 95% to 100%, preferably 98-100%, of the length of the first and second main member is embedded in the ilium.

12. The pelvic fixation device of any one of the claims 1 to 11, wherein the first interlocking member and the second interlocing member is configured such that 95% to 100%, preferably 98-100%, of the length of the first and second interlocking member is embedded in the ilium.

13. The pelvic fixation device of any one of the claims 1 to 12, wherein the first main member and the second main member each define proximate diameter of 12 millimetres at the proximal end and proximate diameter of 6 millimetres at a distal end.

14. The pelvic fixation device of any one of the claims 1 to 13, wherein the first main member and the second main member each define proximate length of between 80 millimetres to 100 millimetres.

15. The pelvic fixation device of any one of the claims 1 to 14, wherein the first interlocking member and the second interlocking member each define proximate length of between 50 millimetres to 70 millimetres.

16. The pelvic fixation device of any one of the claims 1 to 15, wherein the connecting rod define proximate length of between 100 millimetres to 140 millimetres.

17. The pelvic fixation device of any one of the claims 1 to 16, wherein the connecting rod define proximate diameter of between 5.5 millimetres to 7.5 millimetres.

18. The pelvic fixation device of any one of the claims 1 to 17, wherein the first main member is a first main screw, the second main member is a second main screw, the first interlocking member is a first interlocking screw, and the second interlocking member is a second interlocking screw.

19. A method of stabilizing the sacroiliac joint or any one of the bones interfacing with the sacroiliac joint comprising inserting in the subject on the posterior side of the pelvis the first and second pairs of fixation members of the posterior pelvic fixation device according to any one of claims 1 to 18.

20. The method of claim 19, wherein 95% to 100%, preferably 98-100%, of the length of the first and second main members and the first and second interlocking member is inserted in the ilium

21. The method of claim 19 or 20, comprising creating a tunnel in the pelvic bone that allows for the first interlocking member to pass through the hole or slot in the first main screw that is configured to receive the first interlocking member and creating a tunnel in the pelvic bone that allows for the second interlocking member to pass through the hole or slot in the second main screw that is configured to receive the second interlocking member.

22. The method of any one of claims 19 to 21, wherein the device is according to any one of claims 2 to 17 and the method comprises creating a tunnel in the pelvic bone that allows for the connecting rod to pass through the hole in the first main member that is configured to receive the connecting rod and creating a tunnel in the pelvic bone that allows for the connecting rod to pass through the hole in the second main member that is configured to receive the connecting rod.

23. The method of any one of claims 19 to 22, comprising removing the device after the injury has healed or after the sacroiliac joint or any one of the bones interfacing with the sacroiliac joint have been stabilized with a different device.
